(19) United States
(12) Patent Application Publication
(10) Pub. No.: US 2022/0056025 A1
(43) Pub. Date: Feb. 24, 2022

(54) SYNTHESIS OF PYRIDO[2,3-D]PYRIMIDIN-7(8H)-ONES

(71) Applicant: Pfizer Inc., New York, NY (US)

(72) Inventors: Adam Ross Brown, Old Lyme, CT (US); Jean-Nicolas Desrosiers, Guilford, CT (US); Shengquan Duan, East Lyme, CT (US); Joel Michael Hawkins, Lyme, CT (US); Cheryl Myers Hayward, Old Lyme, CT (US); Mark Thomas Maloney, East Lyme, CT (US); Sebastien Monfette, South Windsor, CT (US); Hahdi Hakimioun Perfect, Ledyard, CT (US); Daniel William Widlicka, Ledyard, CT (US)

(73) Assignee: Pfizer Inc., New York, NY (US)

(21) Appl. No.: 17/279,483

(22) PCT Filed: Sep. 23, 2019
(86) PCT No.: PCT/IB2019/058042

§ 371 Date: Mar. 24, 2021

Related U.S. Application Data
(60) Provisional application No. 62/892,884, filed on Aug. 28, 2019.
(60) Provisional application No. 62/870,462, filed on Jul. 3, 2019.
(60) Provisional application No. 62/736,010, filed on Sep. 25, 2018.

(51) Int. Cl. C07D 471/04; B01J 23/44

(52) U.S. Cl. CPC C07D 471/04; B01J 23/44

(57) ABSTRACT

This invention relates to novel methods to prepare substituted pyrido[2,3-d]pyrimidin-7(8H)-ones, and salts and stereoisomers thereof, as well as intermediates useful for the preparation of such compounds.

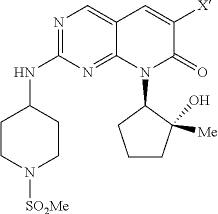

### BACKGROUND OF THE INVENTION

### Field of the Invention

The present invention relates to novel methods to prepare substituted pyrido[2,3-d]pyrimidin-7(8H)-ones, and salts and stereoisomers thereof. The invention further provides intermediates useful for the preparation of such compounds.

### Description of the Related Art

Cyclin-dependent kinases (CDKs) are important cellular enzymes that perform essential functions in regulating eukaryotic cell division and proliferation. CDK inhibitors may be useful for the treatment of proliferative disorders, including cancer.

Substituted pyrido[2,3-d]pyrimidin-7(8H)-one derivatives useful as CDK2/4/6 inhibitors are disclosed in U.S. Pat. No. 10,233,188 and International Publication No. WO 2018/033815, the contents of which are incorporated herein by reference in their entirety. The synthetic routes described in the above-cited application were not designed for large scale synthesis or commercial scale-up. Therefore, alternative routes for the preparation of such compounds that are cost-efficient, scaleable and productive are highly desirable.

### BRIEF SUMMARY OF THE INVENTION

The present invention provides improved methods to prepare a substituted pyrido[2,3-d]pyrimidin-7(8H)-one compound of Formula (I),

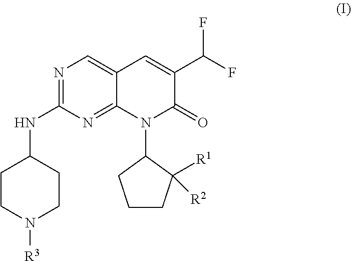

or a pharmaceutically acceptable salt or stereoisomer thereof,

wherein R¹ and R² are independently H, OH, OR⁴ or C₁-C₄ alkyl, provided at least one of R¹ and R² is not H;

R³ is SO₂R⁵ or an amino protecting group;

R⁴ is a hydroxyl protecting group; and

R⁵ is C₁-C₄ alkyl.

The invention further provides methods to prepare 6-(difluoromethyl)-8-[(1R,2R)-2-hydroxy-2-methylcyclopentyl]-2-{[1-(methylsulfonyl)-piperidin-4-yl]amino}pyrido[2,3-d]pyrimidin-7(8H)-one (PF-06873600), having the structure of Formula 1:

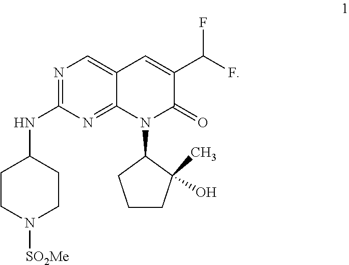 [SMILES: C[C@@]1(O)CCC[C@H]1n1c(=O)c(C(F)F)cc2cnc(NC3CCN(S(C)(=O)=O)CC3)nc21]

The compound of Formula 1 is a CDK2/4/6 inhibitor disclosed in example 10 of U.S. Pat. No. 10,233,188.

In one aspect, the invention provides a method for preparing the compound of Formula 1,

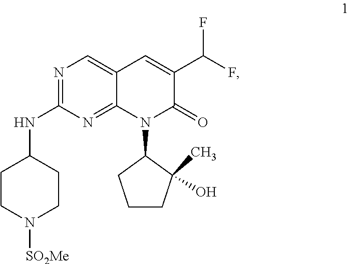 [SMILES: C[C@@]1(O)CCC[C@H]1n1c(=O)c(C(F)F)cc2cnc(NC3CCN(S(C)(=O)=O)CC3)nc21]

comprising reacting a compound of Formula 5a:

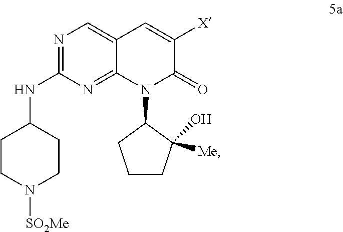

where X′ is Cl, Br, I, OTf or OTs,

with a difluoromethylation agent and a copper reagent to provide the compound of Formula 1.

In some embodiments, X′ is Cl, Br or I. In some embodiments, X′ is Br or I. In some such embodiments, X′ is Br. In other such embodiments, X′ is I. In other embodiments, X′ is Cl. In other embodiments, X′ is OTf or OTs. In some such embodiments, X′ is OTf. In other such embodiments, X′ is OTs.

Suitable copper reagents include copper(I) or copper(II) reagents and complexes.

In some embodiments, the difluoromethylation agent is a copper difluoromethyl complex or a zinc difluoromethyl complex. In some such embodiments, the difluoromethylation agent is a copper difluoromethyl complex. In other such embodiments, the difluoromethylation agent is a zinc difluoromethyl complex. In some such embodiments, the copper or zinc difluoromethyl complexes are prepared separately. In other such embodiments, the copper or zinc difluoromethyl complexes are prepared in situ.

The invention further provides intermediates useful for preparing the compound of Formula 1, or a salt thereof.

### DETAILED DESCRIPTION OF THE INVENTION

The present invention may be understood more readily by reference to the following detailed description of the preferred embodiments of the invention and the Examples included herein. It is to be understood that the terminology used herein is for the purpose of describing specific embodiments only and is not intended to be limiting. It is further to be understood that unless specifically defined herein, the terminology used herein is to be given its traditional meaning as known in the relevant art.

As used herein, the singular form “a”, “an”, and “the” include plural references unless indicated otherwise. For example, “a” substituent includes one or more substituents.

The invention described herein suitably may be practiced in the absence of any element(s) not specifically disclosed herein. Thus, for example, in each instance herein any of the terms “comprising”, “consisting essentially of”, and “consisting of” may be replaced with either of the other two terms.

The term “alkoxide base,” as used herein, refers to M⁺OR″, wherein M⁺ is a cation selected from the group consisting of lithium, sodium, potassium and cesium, and R″ is C₁-C₅ alkyl, as defined herein. Examples of alkoxide bases include lithium methoxide, lithium ethoxide, lithium isopropoxide, lithium tert-butoxide, sodium methoxide, sodium ethoxide, sodium isopropoxide, sodium tert-butoxide, sodium tert-pentoxide, potassium methoxide, potassium ethoxide, potassium isopropoxide, potassium tert-butoxide, potassium tert-pentoxide, and the like.

The term “alkyl,” as used herein, refers to a saturated, monovalent straight or branched chain hydrocarbon having from one to six carbons (C₁-C₆ alkyl), sometimes from one to five carbons (C₁-C₅ alkyl), and preferably from one to four carbons (C₁-C₄ alkyl). Representative examples of alkyl groups are methyl, ethyl, n-propyl, isopropyl, n-butyl, isobutyl, sec-butyl, tert-butyl, and the like.

The term “amino protecting group,” as used herein, refers to selectively introducible and removable groups which protect amino groups against undesirable side reactions during synthetic procedures. Representative examples of amino protecting groups include carbamates (e.g., carbobenzyloxy (Cbz), tert-butyloxycarbonyl (Boc) or fluorenylmethyloxycarbonyl (Fmoc)), amides (e.g., acetamide, trifluoroacetamide or formamide), sulfonamides (e.g., tosylamide) and benzylic groups (e.g., benzyl, p-methoxybenzyl (PMB) or 3,4-dimethoxybenzyl (DMPM)). Such amino protecting groups could be useful to replace R³ in the compounds and methods described herein. In some embodiments, the amino protecting group is selected from the group consisting of a carbamate, an amide, a sulfonamide, and a benzylic group optionally substituted by one or more methoxy substituents. In some such embodiments, the carbamate is CBz, Boc or Fmoc; the amide is acetamide, trifluoroacetamide or formamide; the sulfonamide is tosylamide; and the benzylic group is benzyl, PMB or DMPM.

Some of the methods described herein include a copper reagent. Suitable copper reagents include copper(I) or copper(II) reagents and complexes. Examples of suitable copper reagents include copper(I) chloride (CuCl), copper(I) iodide (CuI), copper(I) trifluoromethanesulfonate (CuOTf), copper(II) trifluoromethanesulfonate (Cu(OTf)₂), tetrakis(acetonitrile)copper(l)tetrafluoroborate (Cu(BF₄)(MeCN)₄) or tetrakis(aceto-nitrile)copper(I) hexafluorophosphate (Cu(PF₆)(MeCN)₄).

The term “halo,” as used herein refers to Cl, Br or I.

The term “hydroxyl,” as used herein, refers to —OH.

The term “hydroxyl protecting group,” as used herein, refers to selectively introducible and removable groups which protect hydroxyl groups against undesirable side reactions during synthetic procedures. Representative examples of hydroxyl protecting groups include ethers (e.g., benzyl, trityl or trialkylsilyl ethers), esters (e.g., acetyl or benzoyl) and acetals (e.g., tetrahydropyranyl ethers). Such hydroxyl protecting groups could be useful to replace R⁴ in the compounds and methods described herein. In some embodiments, the hydroxyl protecting group is selected from the group consisting of an ether, an ester, and an acetal. In some such embodiments, the ether is a benzyl, trityl or trialkylsilyl ether (e.g., TMS, TES, TBDMS); the ester is an acetyl or benzoyl ester; and the acetal is a tetrahydropyranyl ether.

The term “OTf” as used herein refers to a trifluoromethanesulfonate ester or triflate ester (i.e., —OSO₂CF₃) moiety.

The term “OTs” as used herein refers to a p-toluenesulfonate ester or tosylate ester (i.e., —OSO₂C₆H₄CH₃) moiety.

The term “protecting group,” as used herein, refers to selectively introducible and removable groups which protect functional groups against undesirable side reactions during synthetic procedures. Examples of suitable protecting groups for various functional groups and relevant reaction conditions are provided in Wuts, Peter G. M. Greene's Protective Groups in Organic Synthesis (5^{th} ed.). New York: Wiley, 2007.

As described herein, some reactions are optionally run in the presence of a protic source. Depending on the nature of the chemical reaction, such reagents may be present in catalytic, substoichiometric or stoichiometric amounts and may accelerate the rate of reaction or increase the extent of the conversion. It is typically possible to run such reactions in the absence of the protic source, particularly on small scale.

In some embodiments, the protic source comprises a carboxylic acid, sulfonic acid, sulfinic acid, alcohol, thiol or primary amine. In some embodiments, the protic source comprises a carboxylic acid or a sulfonic acid, e.g., p-toluenesulfonic acid or oxalic acid. In other embodiments, the protic source comprises a sulfinic acid, an alcohol, a thiol or a primary amine, e.g., p-toluenesulfinic acid, water, propylene glycol or pinacol. When used as a catalyst, the amount of the protic source may range from about 0.01 to about 0.30 molar equivalents (i.e., about 1% to about 30%), and frequently from about 0.05 to about 0.15 molar equivalents (i.e., about 5% to about 15%). In some embodiments, the protic source is present in an amount of about 0.25, about 0.2, about 0.15, about 0.10, or less than about 0.10 molar equivalents. In other reactions, a protic source may be present in substoichiometric or stoichiometric amounts, e.g., from about 0.50 to about 1.0 molar equivalents or greater, and typically from about 0.70 to about 1.0 molar equivalents or greater.

In one aspect, the invention provides a general three-step method, illustrated in Scheme A, for preparing intermediate compounds of Formula 5a.

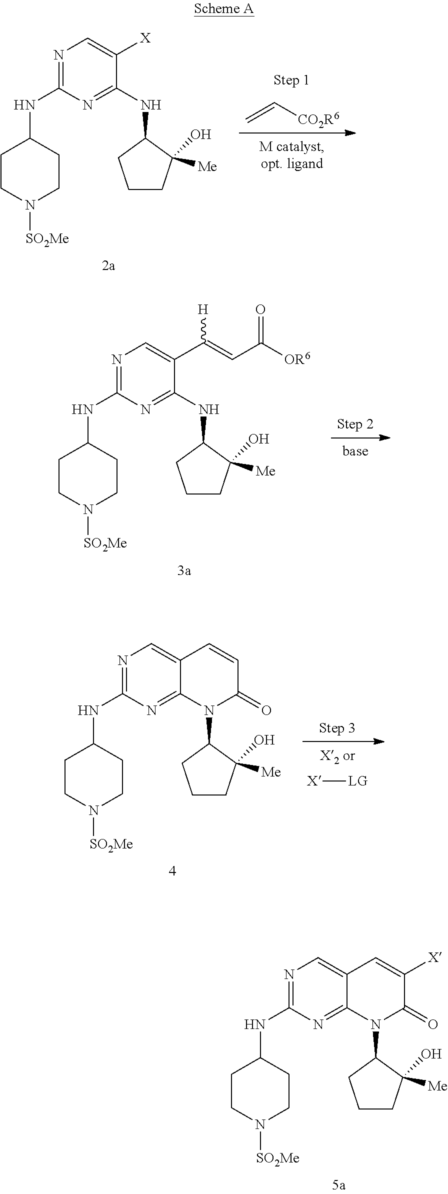

According to Step 1 of Scheme A, the compound of Formula 3a is prepared by reacting the intermediate of Formula 2a (where X is Cl, Br, I, OTf or OTs, prepared by reaction of intermediate 1b described in Example 7/Example 8 of U.S. Pat. No. 10,233,188 with a suitable 5-substituted-2,6-dichloropyrimidine) with an alkyl or benzyl acrylate (i.e., R⁶ is C₁-C₄ alkyl or benzyl) in the presence of a metal (“M”) catalyst, such as a palladium, copper, nickel, cobalt or iron catalyst. Preferably, the catalyst is a palladium (Pd) catalyst. In some embodiments, the catalyst is a Pd(II) catalyst. In one preferred embodiment, the catalyst is palladium(II) acetate (i.e., Pd(OAc)₂). In other embodiments, the catalyst is a Pd(0) catalyst. The metal catalyst is typically present in an amount from about 0.01 to about 0.10 molar equivalents relative to intermediate 2a. Optionally, the coupling reaction includes a ligand, such as a phosphine ligand. When used, the phosphine ligand is typically present in an amount from about 0.01 to about 0.10 molar equivalents.

Intermediate compounds of Formula 3a contain predominantly the trans geometric isomer (E-olefin) but may contain varying amounts of the cis geometric isomer (Z-olefin). The compounds of Formula 3a may be purified (e.g., chromatography or crystallization) or the crude mixture after aqueous work-up may be directly used in the subsequent cyclization (Step 2) without further purification. In some embodiments, the compound of Formula 3a is isolated as the E-olefin. However, it is not necessary to separate the E- and Z-olefinic mixture before cyclization.

According to Step 2 of Scheme A, the compound of Formula 4 is prepared by cyclization of compound 3a under basic conditions. The compound of Formula 4 was previously described in Example 2 of U.S. Pat. No. 10,233,188. Preferred bases for use in Step 2 are alkoxide bases, preferably methoxide, ethoxide or t-butoxide bases. The alkoxide base is typically present in an amount from about 1.0 to about 5.0 molar equivalents relative to intermediate 3a. The compound of Formula 4 may be purified (e.g., by crystallization) or may be isolated and used in the subsequent halogenation reaction (Step 3) without further purification.

According to Step 3 of Scheme A, the compound of Formula 5a is prepared by halogenation of the compound Formula 4 under electrophilic conditions, to provide 5a where X′ is Cl, Br or I. When X′ is iodo, the preferred iodination reagents are iodine or N-iodosuccinimide (NIS). When X′ is bromo, the preferred bromination reagents are bromine or N-bromosuccinimide (NBS). When X′ is chloro, the preferred chlorination reagent is N-chlorosuccinimide (NCS). Other suitable halogenation reagents are known to those of skill in the art, including, e.g., 1,3-diiodo-5,5′-dimethylhydantoin (DIH), N-iodophthalimide, N-bromophthalimide, and N-chlorophthalimide. When the halogenation reagent is NIS, NBS or NCS, the leaving group “LG” is the succinimide moiety. Similarly, the leaving groups for DIH and the N-halophthalimides are 5,5′-dimethylhydantoin and phthalimide, respectively. The halogenation reagent may be present in a stoichiometric amount or in excess relative to intermediate 4, for example from about 1.0 to about 2.0 molar equivalents, and sometimes about 1.5 molar equivalents.

The halogenation reactions in Step 3 typically contain a catalytic amount of a protic source. In some embodiments, the protic source comprises a carboxylic acid or a sulfonic acid. In some such embodiments, the protic source is p-toluenesulfonic acid or oxalic acid. In some embodiments, the protic source.is present in an amount from about 0.01 to about 0.30 molar equivalents relative to intermediate 4, and preferably from about 0.05 to about 0.15 molar equivalents. In some embodiments, the protic source. is present in about 0.10 molar equivalents relative to intermediate 4.

In one aspect, the invention provides a method for preparing the compound of Formula 5a according to Scheme A:

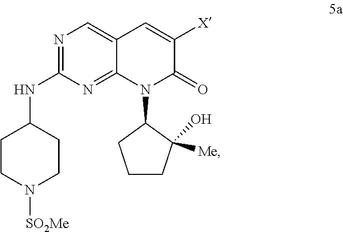

where X′ is Br or I,

comprising the steps of:

(1) preparing the compound of Formula 3a:

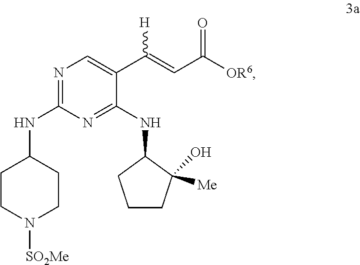

wherein R⁶ is C₁-C₄ alkyl or benzyl,

comprising treating the compound of Formula 2a:

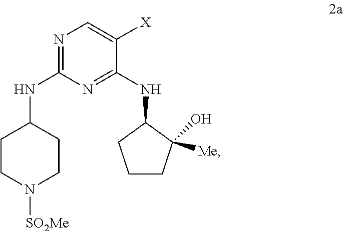

where X is Cl, Br, I, OTf or OTs,

with a C₁-C₄ alkyl acrylate or benzyl acrylate in the presence of a palladium catalyst to provide the compound of Formula 3a;

(2) preparing the compound of Formula 4:

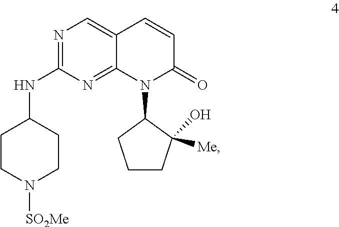 [SMILES: C[C@@]1(O)CCC[C@H]1n1c(=O)ccc2cnc(NC3CCN(S(C)(=O)=O)CC3)nc21]

comprising treating the compound of Formula 3a:

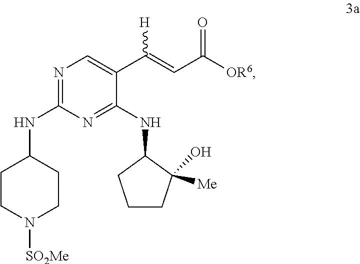

where R⁶ is C₁-C₄ alkyl or benzyl,

with a base to provide the compound of Formula 4; and

(3) treating the compound of Formula 4:

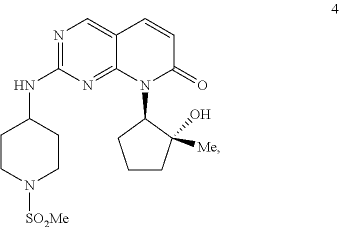 [SMILES: C[C@@]1(O)CCC[C@H]1n1c(=O)ccc2cnc(NC3CCN(S(C)(=O)=O)CC3)nc21]

(i) with bromine or N-bromosuccinimide to provide the compound of Formula 5a where X′ is Br; or

(ii) with iodine or N-iodosuccinimide to provide the compound of Formula 5a where X′ is I.

In some embodiments, the method further comprises a step (4) for preparing the compound of Formula 1 from 5a (where X′ is I) according to Scheme C.

The palladium catalyst in step (1) is selected as further described herein. is palladium acetate and optionally a ligand. In some such embodiments, the palladium catalyst is palladium acetate. In some embodiments, the base in step (2) is an alkoxide base as further described herein. In some embodiments, the halogenation reaction in step (3) is run in the presence of a protic source as further described herein. All reactions are run in suitable solvent and temperature, as described.

Scheme B illustrates a specific method of preparing the iodo-intermediate compound of Formula 5b according to the three-step sequence outlined above.

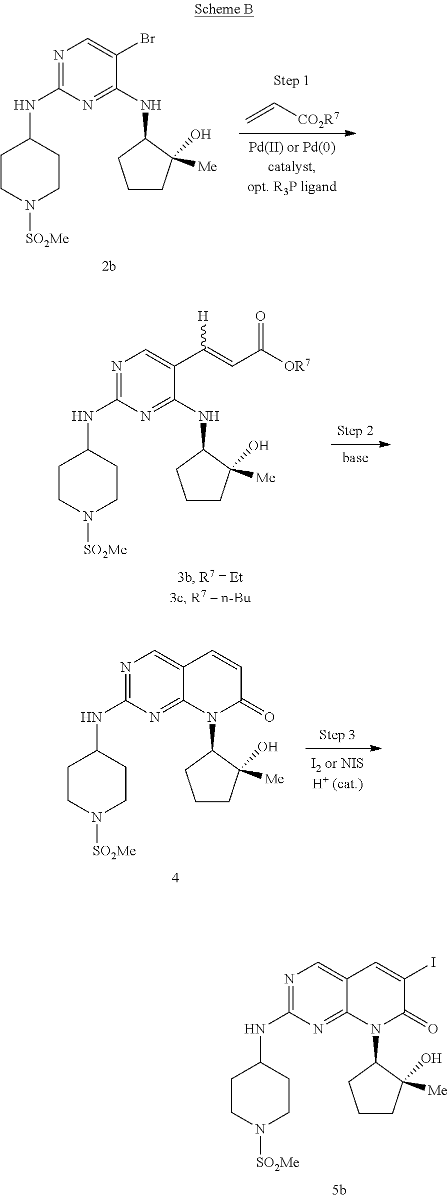

According to Step 1 of Scheme B, compounds of Formula 3b or 3c are prepared by treating the compound of Formula 2b (prepared as described in Example 7/Example 8 of U.S. Pat. No. 10,233,188) with ethyl acrylate or n-butyl acrylate, respectively, in the presence of a palladium catalyst, preferably a Pd(II) catalyst such as Pd(OAc)₂. The palladium catalyst is typically present in an amount from about 0.01 to about 0.10 molar equivalents relative to intermediate 2b. The compounds of Formula 3b and 3c are prepared predominantly as the trans geometric isomer but may contain varying amounts of the cis geometric isomer.

Optionally, the coupling reaction includes a ligand, such as a phosphine ligand. In some such embodiments, the phosphine ligand is selected from the group consisting of n-butyl-di-t-butylphosphonium tetraborofluorate, 1,4-bis(di-t-butylphosphonium)butane bis(tetrafluoroborate), triphenylphosphine, cyclohexyldiphenylphosphine, (oxydi-2,1-phenylene)-bis(diphenylphosphine) (DPEPhos), (oxydi-2,1-phenylene)bis(dicyclohexyl-phosphine) (DCyEPhos), 1,3-bis(diphenylphosphino)propane (dppp), 1,4-Bis(diphenyl-phosphino)-butane (dppb), di-(1-adamantyl)-n-butylphosphine (CataCXium® A), bis(di-tert-butyl(4-dimethylaminophenyl)phosphine (Amphos), 5-(di-tert-butylphosphino)-1′,3′,5′-triphenyl-1′H-[1,4′]bipyrazole (Bippyphos), 1,1′-bis(di-tert-butylphosphino)-ferrocene (DTBPF), 1,3-bis(2,6-diisopropylphenyl)imidazolinium chloride (SIPr—HCl) and 1,3-Bis(1-adamantyl)-4,5-dihydroimidazolium chloride (Sad-HCl). When used, the phosphine ligands are typically present in an amount from about 0.01 to about 0.10 molar equivalents.

In another aspect, the invention provides a method for preparing the compound of Formula 5b according to Scheme B:

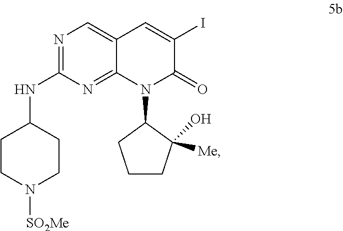 [SMILES: C[C@@]1(O)CCC[C@H]1n1c(=O)c(I)cc2cnc(NC3CCN(S(C)(=O)=O)CC3)nc21]

comprising the steps of:

(1) preparing the compound of Formula 3b or 3c:

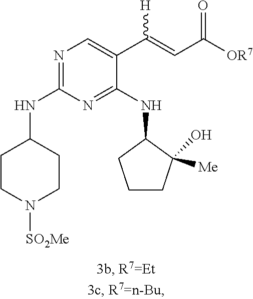

comprising treating the compound of Formula 2b:

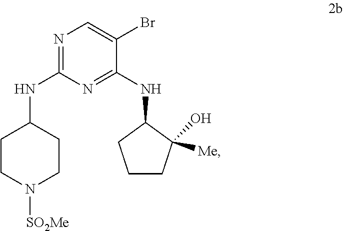 [SMILES: C[C@@]1(O)CCC[C@H]1Nc1nc(NC2CCN(S(C)(=O)=O)CC2)ncc1Br]

with ethyl acrylate or n-butyl acrylate in the presence of a palladium catalyst to provide the compound of Formula 3b or 3c;

(2) preparing the compound of Formula 4:

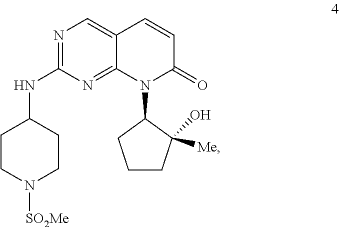 [SMILES: C[C@@]1(O)CCC[C@H]1n1c(=O)ccc2cnc(NC3CCN(S(C)(=O)=O)CC3)nc21]

comprising treating the compound of Formula 3b or 3c:

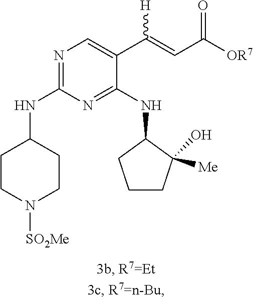

with a base to provide the compound of Formula 4; and

(3) treating the compound of Formula 4:

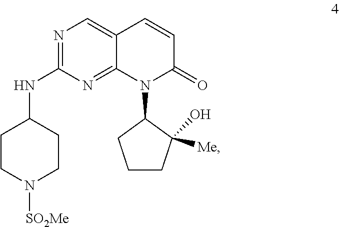 [SMILES: C[C@@]1(O)CCC[C@H]1n1c(=O)ccc2cnc(NC3CCN(S(C)(=O)=O)CC3)nc21]

with iodine or N-iodosuccinimide to provide the compound of Formula 5b.

In some embodiments, the method further comprises a step (4) for preparing the compound of Formula 1 from 5b according to Scheme C.

The palladium catalyst in step (1) is selected as further described herein. Step (1) optionally comprises a ligand, such as a phosphine ligand. In some such embodiments, the palladium catalyst is palladium acetate. In some embodiments, the base in step (2) is an alkoxide base as further described herein. In some embodiments, the halogenation reaction in step (3) is run in the presence of a protic source as further described herein. All reactions are run in suitable solvent and temperature, as described.

Scheme C illustrates two methods (Method A and Method B) for preparing the compound of Formula 1 by difluoromethylation of the compound of Formula 5b.

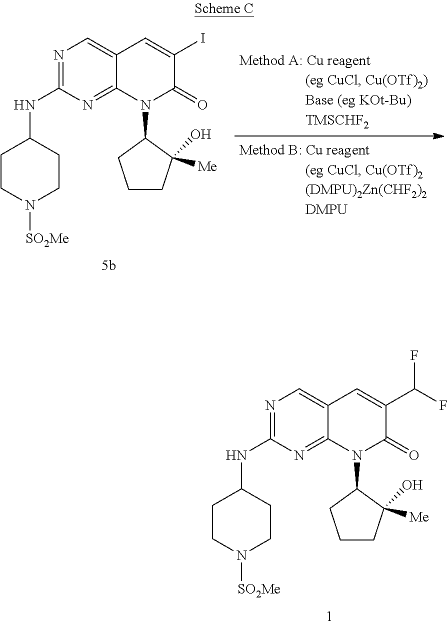 [SMILES: C[C@@]1(O)CCC[C@H]1n1c(=O)c(I)cc2cnc(NC3CCN(S(C)(=O)=O)CC3)nc21 ; C[C@@]1(O)CCC[C@H]1n1c(=O)c(C(F)F)cc2cnc(NC3CCN(S(C)(=O)=O)CC3)nc21]

According to Method A, the compound of Formula 1 is prepared by reacting the compound of Formula 5b with a difluoromethyltrialkylsilane, preferably difluoromethyltrimethylsilane (TMSCHF₂), in the presence of a copper(I) or copper(II) reagent, for example CuCl or Cu(OTf)₂, and a base. Preferably, the base is an alkoxide base, such as potassium t-butoxide (KOt-Bu). Other suitable bases and copper reagents may be used.

In a preferred embodiment of Method A, the copper reagent is combined with the base in an appropriate solvent and the reaction mixture is maintained for an appropriate time and temperature, e.g., approximately 0.5 hours at around 20-30° C., prior to addition of the difluoromethyltrialkylsilane reagent, followed by addition of the compound of Formula 5b.

Preferred solvents for Method A include polar aprotic solvents, such as N,N′ dimethylpropyleneurea (DMPU), N,N′-dimethylformamide (DMF), or mixtures thereof, or mixtures of DMF and/or DMPU with other organic solvents. For Method A, the stoichiometry of the copper reagent to base ranges from about 1:1 to about 1:3, and typically is about 1:2. The stoichiometry of the copper reagent to the difluoromethyltrialkylsilane ranges from about 1:1 to about 1:3, and frequently is about 1:2. The stoichiometry of the copper reagent to the compound of Formula 5b should be not less than 1:1, and preferably an excess amount of the copper reagent is used. In some embodiments, the copper reagent may be used in an amount of about 1.0 molar equivalents to about 3.0 molar equivalents with respect to the compound of Formula 5b. In some such embodiments, the stoichiometry of the copper reagent to 5b is about 1.5:1, about 2:1 or about 3:1. Frequently, the stoichiometry of the copper reagent to 5b is about 1.5:1. In some embodiments, the reaction comprises about 3 equivalents base, about 1.5 equivalents copper reagent, and about 2.5 to about 3.5 equivalents of difluoromethyltrialkylsilane, in each case relative to 1.0 molar equivalents of 5b.

According to Method B, the compound of Formula 1 is prepared by reacting the compound of Formula 5b with a zinc difluoromethyl complex, Zn(DMPU)₂(CHF₂)₂, in the presence of a copper(I) or copper(II) reagent, for example CuCl, CuOTf or Cu(OTf)₂.

Preferred solvents for Method B include polar aprotic solvents such as DMPU, DMF, or mixtures thereof, or mixtures of DMF and/or DMPU with other organic solvents, with DMPU particularly preferred.

For Method B, the stoichiometry of the copper reagent to 5b ranges from about 0.5 to about 1.5 molar equivalents, and sometimes about 0.9 molar equivalents. The stoichiometry of the zinc difluoromethyl complex to 5b ranges from about 1.0 to about 5.0 molar equivalents, and sometimes about 3.0 molar equivalents. In some embodiments, the reaction comprises about 0.9 equivalents copper reagent and about 3.0 equivalents of zinc difluoromethyl complex, in each case relative to 1.0 molar equivalents of 5b.

In some embodiments, the difluoromethylation reactions are run in the presence of a protic source. In some embodiments or Method A and Method B, the protic source is p-toluenesulfinic acid, water, propylene glycol or pinacol. In some embodiments of Method A, the protic source is propylene glycol in an amount of from about 0.65 to about 0.85 molar equivalents, preferably from about 0.70 to about 0.75 molar equivalents, relative to 5b. In some embodiments of Method B, the protic source is propylene glycol or p-toluenesulfinic acid in an amount of about 0.20 to about 0.30 molar equivalents, preferably about 0.25 molar equivalents, relative to 5b.

In some embodiments, the invention provides the method for preparing the compound of Formula 1 according to Scheme C, wherein 5b is prepared according to Steps 1 to 3 of Scheme A or Scheme B.

Scheme D illustrates the process for preparing the zinc complex, Zn(DMPU)₂(CHF₂)₂.

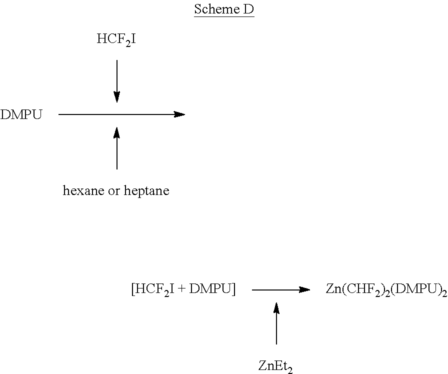

The zinc complex, Zn(DMPU)₂(CHF₂)₂, may be prepared by treating iododifluoromethane (HCF₂I) with diethyl zinc (ZnEt₂), preferably by a continuous or semi-continuous process. In one embodiment, iododifluoromethane, diethyl zinc, and DMPU are combined simultaneously. The zinc reagent may be prepared in batch mode or may be prepared using flow chemistry under an inert atmosphere.

In one embodiment, the compound of Formula 5b is treated with continuously or semi-continuously prepared Zn(DMPU)₂(CHF₂)₂ in the presence of the copper reagent to provide the compound of Formula 1 in a contiguous continuous or semi-continuous process, respectively.

In one aspect, the invention provides a method for preparing the compound of Formula 1,

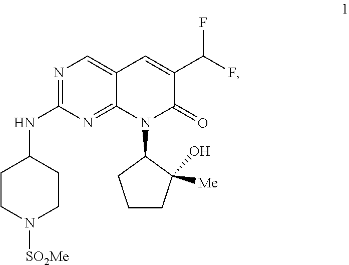 [SMILES: C[C@@]1(O)CCC[C@H]1n1c(=O)c(C(F)F)cc2cnc(NC3CCN(S(C)(=O)=O)CC3)nc21]

comprising reacting a compound of Formula 5a:

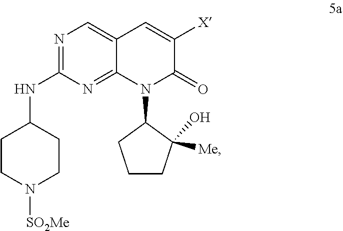

wherein X′ is Cl, Br, I, OTf or OTs,

with a difluoromethylation agent and a copper reagent to provide the compound of Formula 1.

In some embodiments of this aspect, X′ is Cl, Br or I. In frequent embodiments of this aspect, X′ is I. In other embodiments, X′ is Br or Cl. In other embodiments, X′ is Br. In still other embodiments, X′ is Cl. In further embodiments, X′ is OTf or OTs.

In some embodiments of this aspect, the difluoromethylation agent is a difluoromethyltrialkylsilane. In specific embodiments, the difluoromethyltrialkylsilane is difluoromethyltrimethylsilane (TMSCHF₂).

Embodiments using a difluoromethyltrialkylsilane are typically conducted in the presence of a suitable base, for example an alkoxide base such as potassium tert-butoxide or other suitable alkoxide base as described herein. In some embodiments, the reaction of 5a with the difluoromethyltrialkylsilane and the copper reagent further comprises a base, in particular an alkoxide base.

Embodiments using a difluoromethyltrialkylsilane are typically conducted in the presence of a protic source. In some embodiments, the reaction of 5a with the difluoromethyltrialkylsilane and the copper reagent further comprises a protic source. In some such embodiments, the protic source comprises a sulfinic acid, an alcohol, a thiol or a primary amine. In some such embodiments, the protic source is p-toluenesulfinic acid, water, propylene glycol or pinacol. In other embodiments, the protic source comprises an alcohol, thiol or primary amine. In specific embodiments, the protic source is water, propylene glycol or pinacol. In further embodiments, the protic source comprises a sulfinic acid. In some such embodiments, the protic source is p-toluenesulfinic acid. In some embodiments, the reaction is conducted in the presence of a catalytic amount of the protic source. In some such embodiments, the reaction is conducted in the presence of a catalytic amount of p-toluenesulfinic acid, water, propylene glycol or pinacol.

In some embodiments, the reaction of 5a with the difluoromethyltrialkylsilane and the copper reagent further comprises a base and a protic source, as further described herein.

In some embodiments, reaction of the difluoromethyltrialkylsilane reagent and a copper(I) reagent in the presence of base may form a copper difluoromethyl complex in situ, which acts as the difluoromethylation agent.

In other embodiments, the difluoromethylation agent is a zinc difluoromethyl complex. In certain preferred embodiments, the zinc difluoromethyl complex is Zn(CHF₂)₂(DMPU)₂.

In some embodiments, the reaction of 5a with a zinc difluoromethyl complex is conducted in the presence of a protic source. In some such embodiments, the protic source comprises a sulfinic acid, an alcohol, a thiol or a primary amine. In some such embodiments, the protic source is p-toluenesulfinic acid, water, propylene glycol or pinacol. In some such embodiments, the protic source comprises a sulfinic acid or an alcohol. In specific embodiments, the protic source is p-toluenesulfinic acid or propylene glycol. In some such embodiments, the reaction is conducted in the presence of a catalytic amount of the protic source. In some such embodiments, the reaction is conducted in the presence of a catalytic amount of p-toluenesulfinic acid, water, propylene glycol or pinacol.

In some embodiments of this aspect, the copper reagent is a copper(I) reagent or a copper(II) reagent. In some embodiments, the copper reagent is CuCl, CuI, Cu(OTf), Cu(OTf)₂, Cu(BF₄)(MeCN)₄ or Cu(PF₆)(MeCN)₄. In some embodiments, the copper reagent is CuCl, CuI, CuOTf or Cu(OTf)₂.

In some embodiments, the copper reagent is a copper(I) reagent. In some such embodiments, the copper(I) reagent is CuCl, CuI, Cu(OTf), Cu(BF₄)(MeCN)₄ or Cu(PF₆)(MeCN)₄. In other such embodiments, the copper(I) reagent is CuCl, CuI or CuOTf. In some such embodiments, the copper(I) reagent is CuCl. In other such embodiments, the copper(I) reagent is CuI. In other such embodiments, the copper(I) reagent is Cu(OTf). In still other such embodiments, the copper(I) reagent is Cu(BF₄)(MeCN)₄ or Cu(PF₆)(MeCN)₄.

In other embodiments, the copper reagent is a copper(II) reagent. In some such embodiments, the copper(II) reagent is Cu(OTf)₂.

In some embodiments, the reaction is conducted in the presence of a catalytic or substoichiometric amount of the copper(I) or copper (II) reagent. In some such embodiments, the reaction is conducted in the presence of a catalytic amount of the copper(I) or copper (II) reagent. In some embodiments, the reaction is conducted in the presence of a substoichiometric amount of the copper(I) or copper (II) reagent.

Difluoromethylation reactions are carried out in a suitable solvent or mixture of solvents. Preferred solvents include polar aprotic solvents such as DMPU, DMF, or mixtures thereof, or mixtures of DMF and/or DMPU with other organic solvents, with DMPU particularly preferred. In frequent embodiments, the solvent comprises DMPU, DMF, or mixtures thereof, or mixtures of DMF and/or DMPU with other organic solvents.

In some embodiments, the solvent is DMF. In other embodiments, the solvent is DMPU. In some embodiments, the solvent is a mixture of DMF and DMPU. In further embodiments, the solvent is a mixture of DMF and/or DMPU with one or more other organic solvents. In some embodiments, the solvent comprises DMF. In other embodiments, the solvent comprises DMPU. In other embodiments, the solvent comprises DMPU and DMF. In further embodiments, the solvent comprises a mixture of DMF and/or DMPU with one or more other organic solvents.

In another aspect, the invention provides a method for preparing the compound of Formula 1,

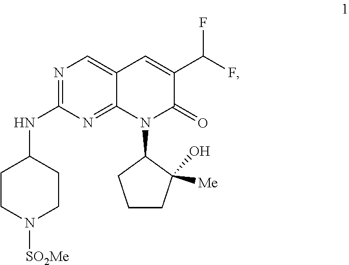 [SMILES: C[C@@]1(O)CCC[C@H]1n1c(=O)c(C(F)F)cc2cnc(NC3CCN(S(C)(=O)=O)CC3)nc21]

comprising reacting a compound of Formula 5b:

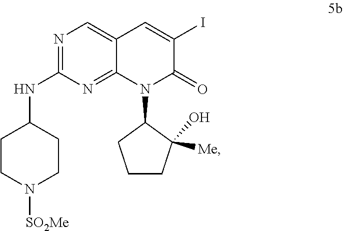 [SMILES: C[C@@]1(O)CCC[C@H]1n1c(=O)c(I)cc2cnc(NC3CCN(S(C)(=O)=O)CC3)nc21]

with a difluoromethyltrialkylsilane, a copper reagent and a base, to provide the compound of Formula 1.

In some embodiments, the difluoromethyltrialkylsilane is TMSCHF₂.

In embodiments of this aspect, the reaction of 5b with the difluoromethyltrialkylsilane is conducted in the presence of a suitable base, for example an alkoxide base such as potassium tert-butoxide or other suitable alkoxide base as described herein. In some such embodiments, the alkoxide base is potassium tert-butoxide.

In some such embodiments of this aspect, the difluoromethylation reaction is conducted in the presence of a protic source. In some embodiments, the reaction of 5b with the difluoromethyltrialkylsilane, copper reagent and base further comprises a protic source. In some such embodiments, the protic source comprises a sulfinic acid, an alcohol, a thiol or a primary amine. In some such embodiments, the protic source is p-toluenesulfinic acid, water, propylene glycol or pinacol. In particular embodiments, the protic source is propylene glycol, pinacol or water. In other embodiments, the protic source is p-toluenesulfinic acid.

In some embodiments of this aspect, the copper reagent is a copper(I) reagent or a copper(II) reagent. In some embodiments, the copper reagent is CuCl, CuI, CuOTf or Cu(OTf)₂. In some embodiments, the copper reagent is CuCl, CuI, Cu(OTf), Cu(OTf)₂, Cu(BF₄)(MeCN)₄ or Cu(PF₆)(MeCN)₄.

In some embodiments, the copper reagent is a copper(I) reagent. In some such embodiments, the copper(I) reagent is CuCl, CuI or CuOTf. In other such embodiments, the copper(I) reagent is CuCl, CuI, Cu(OTf), Cu(BF₄)(MeCN)₄ or Cu(PF₆)(MeCN)₄. In some such embodiments, the copper(I) reagent is CuCl. In other such embodiments, the copper(I) reagent is CuI. In other such embodiments, the copper(I) reagent is Cu(OTf).

In still other such embodiments, the copper(I) reagent is Cu(BF₄)(MeCN)₄ or Cu(PF₆)(MeCN)₄.

In other embodiments, the copper reagent is a copper(II) reagent. In some such embodiments, the copper(II) reagent is Cu(OTf)₂.

In some embodiments, the reaction is conducted in the presence of a catalytic or substoichiometric amount of the copper(I) or copper (II) reagent. In some such embodiments, the reaction is conducted in the presence of a catalytic amount of the copper(I) or copper (II) reagent. In some embodiments, the reaction is conducted in the presence of a substoichiometric amount of the copper(I) or copper (II) reagent. In embodiments of this aspect, the difluoromethylation reaction step is carried out in a suitable solvent or mixture of solvents. In frequent embodiments, the solvent is a polar aprotic solvent, such as DMPU, DMF, or mixtures thereof, or mixtures of DMF and/or DMPU with one or more other organic solvents. In some embodiments, the solvent is DMF. In other embodiments, the solvent is DMPU. In some embodiments, the solvent is a mixture of DMF and DMPU. In further embodiments, the solvent is a mixture of DMF and/or DMPU with one or more other organic solvents. In some embodiments, the solvent comprises DMF. In other embodiments, the solvent comprises DMPU. In other embodiments, the solvent comprises DMPU and DMF. In further embodiments, the solvent comprises a mixture of DMF and/or DMPU with one or more other organic solvents.

In another aspect, the invention provides a method for preparing the compound of Formula 1,

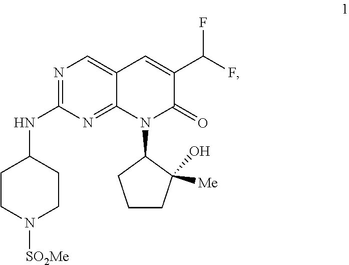 [SMILES: C[C@@]1(O)CCC[C@H]1n1c(=O)c(C(F)F)cc2cnc(NC3CCN(S(C)(=O)=O)CC3)nc21]

comprising reacting a compound of Formula 5b:

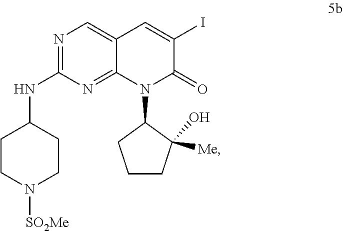 [SMILES: C[C@@]1(O)CCC[C@H]1n1c(=O)c(I)cc2cnc(NC3CCN(S(C)(=O)=O)CC3)nc21]

with a zinc difluoromethyl complex, a copper reagent and a protic source to provide the compound of Formula 1.

In some such embodiments, the zinc difluoromethyl complex is Zn(CHF₂)₂(DMPU)₂. Such complexes may be prepared separately or prepared in situ, as further described herein. In particular embodiments, Zn(DMPU)₂(CHF₂)₂ may be prepared through a continuous or semi-continuous process, for example by treating iododifluoromethane with diethyl zinc and N,N′-dimethylpropyleneurea (DMPU).

In embodiments of this aspect, the reaction of 5b with a zinc difluoromethyl complex is conducted in the presence of a protic source. In some embodiments of this aspect, the protic source comprises a sulfinic acid, an alcohol, a thiol or a primary amine. In some such embodiments, the protic source is p-toluenesulfinic acid, water, propylene glycol or pinacol. In some such embodiments, the protic source comprises a sulfinic acid or an alcohol. In specific embodiments, the protic source is p-toluenesulfinic acid or propylene glycol. In some such embodiments, the reaction is conducted in the presence of a catalytic amount of the protic source. In some such embodiments, the reaction is conducted in the presence of a catalytic amount of p-toluenesulfinic acid, water, propylene glycol or pinacol. In some such embodiments, the reaction is conducted in the presence of a catalytic amount of a protic source, such as p-toluenesulfinic acid or propylene glycol. In some such embodiments, the protic source is p-toluenesulfinic acid.

In some such embodiments, the protic source is propylene glycol.

In some embodiments of this aspect, the copper reagent is a copper(I) reagent or a copper(II) reagent. In some embodiments, the copper reagent is CuCl, CuI, CuOTf or Cu(OTf)₂. In some embodiments, the copper reagent is CuCl, CuI, Cu(OTf), Cu(OTf)₂, Cu(BF₄)(MeCN)₄ or Cu(PF₆)(MeCN)₄.

In some embodiments, the copper reagent is a copper(I) reagent. In some such embodiments, the copper(I) reagent is CuCl, CuI or CuOTf. In other such embodiments, the copper(I) reagent is CuCl, CuI, Cu(OTf), Cu(BF₄)(MeCN)₄ or Cu(PF₆)(MeCN)₄. In some such embodiments, the copper(I) reagent is CuCl. In other such embodiments, the copper(I) reagent is CuI. In other such embodiments, the copper(I) reagent is Cu(OTf). In still other such embodiments, the copper(I) reagent is Cu(BF₄)(MeCN)₄ or Cu(PF₆)(MeCN)₄.

In other embodiments, the copper reagent is a copper(II) reagent. In some such embodiments, the copper(II) reagent is Cu(OTf)₂.

In some embodiments, the reaction is conducted in the presence of a catalytic or substoichiometric amount of the copper(I) or copper (II) reagent. In some such embodiments, the reaction is conducted in the presence of a catalytic amount of the copper(I) or copper (II) reagent. In some embodiments, the reaction is conducted in the presence of a substoichiometric amount of the copper(I) or copper (II) reagent.

In embodiments of this aspect, the difluoromethylation reaction step is carried out in a suitable solvent or mixture of solvents. In frequent embodiments, the solvent is a polar aprotic solvent, such as DMPU, DMF, or mixtures thereof, or mixtures of DMF and/or DMPU with one or more other organic solvents. In frequent embodiments, the solvent comprises DMPU, DMF, or mixtures thereof, or mixtures of DMF and/or DMPU with other organic solvents. In some embodiments, the solvent is DMF. In other embodiments, the solvent is DMPU. In some embodiments, the solvent is a mixture of DMF and DMPU. In further embodiments, the solvent is a mixture of DMF and/or DMPU with one or more other organic solvents. In some embodiments, the solvent comprises DMF. In other embodiments, the solvent comprises DMPU. In other embodiments, the solvent comprises DMPU and DMF. In further embodiments, the solvent comprises a mixture of DMF and/or DMPU with one or more other organic solvents.

In one embodiment, the compound of Formula 5b is treated with the continuously or semi-continuously prepared Zn(DMPU)₂(CHF₂)₂ and an appropriate copper reagent to prepare the compound of Formula 1 in a contiguous continuous or semi-continuous process. In this embodiment, the air and moisture sensitive zinc difluoromethyl complex does not need to be manipulated and/or stored outside of the continuous or semi-continuous processing equipment.

In a further aspect, the invention provides a method for preparing the Zn(DMPU)₂(CHF₂)₂ complex using a continuous or semi-continuous process, comprising treating iododifluoromethane with diethyl zinc and DMPU.

In another aspect, the invention provides a method for preparing the compound of Formula 1:

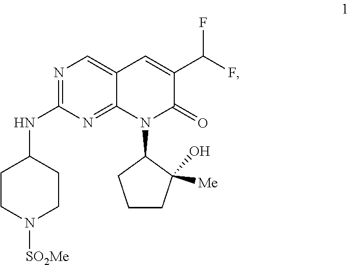 [SMILES: C[C@@]1(O)CCC[C@H]1n1c(=O)c(C(F)F)cc2cnc(NC3CCN(S(C)(=O)=O)CC3)nc21]

comprising reacting the compound of Formula 5b:

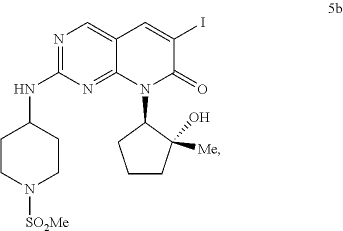 [SMILES: C[C@@]1(O)CCC[C@H]1n1c(=O)c(I)cc2cnc(NC3CCN(S(C)(=O)=O)CC3)nc21]

with continuously or semi-continuously prepared Zn(DMPU)₂(CHF₂)₂ and a copper(I) catalyst in a contiguous continuous or semi-continuous process. In some embodiments, the reaction is conducted in the presence of a protic source, including a catalytic amount of a protic source.

In a further aspect of the invention provides methods for preparing the compound of Formula 1:

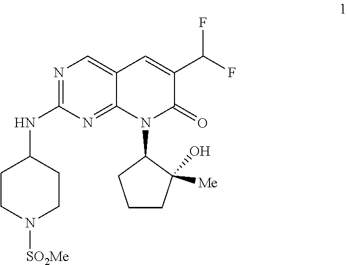 [SMILES: C[C@@]1(O)CCC[C@H]1n1c(=O)c(C(F)F)cc2cnc(NC3CCN(S(C)(=O)=O)CC3)nc21]

comprising treating the compound of Formula 5b,

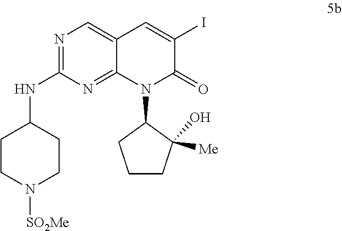 [SMILES: C[C@@]1(O)CCC[C@H]1n1c(=O)c(I)cc2cnc(NC3CCN(S(C)(=O)=O)CC3)nc21]

with a difluoromethylation agent, such as a copper difluoromethyl complex or a zinc difluoromethyl complex, where such complexes may be prepared separately or in situ.

In some embodiments of this aspect, the difluoromethylation agent is a copper difluoromethyl complex. In other embodiments of this aspect, the difluoromethylation agent is a zinc difluoromethyl complex.

In another aspect, the invention provides the compound of Formula 1:

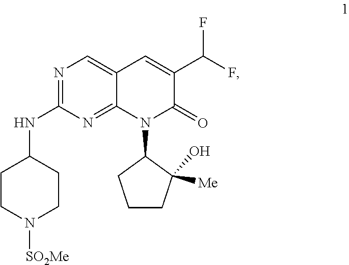 [SMILES: C[C@@]1(O)CCC[C@H]1n1c(=O)c(C(F)F)cc2cnc(NC3CCN(S(C)(=O)=O)CC3)nc21]

prepared according to any of the methods provided herein.

In yet another aspect, the invention provides intermediates useful for the preparation of the compounds described herein. In particular embodiments, the invention provides the following intermediates, which may be useful in the synthesis of the compound of formula 1:

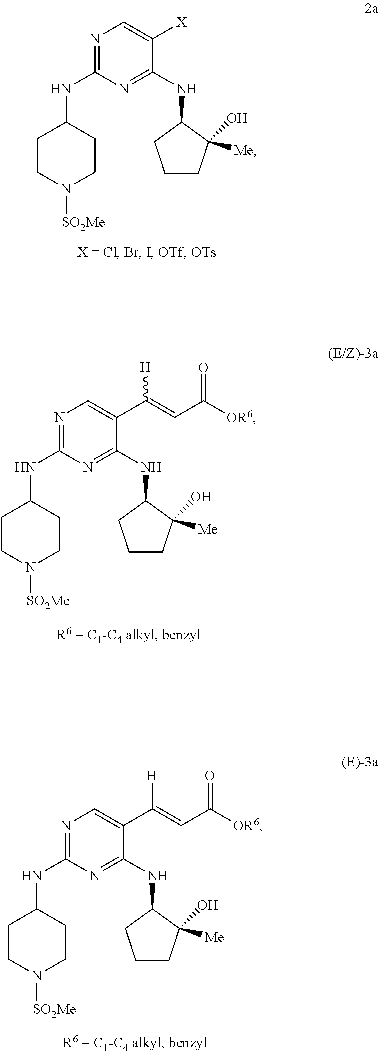

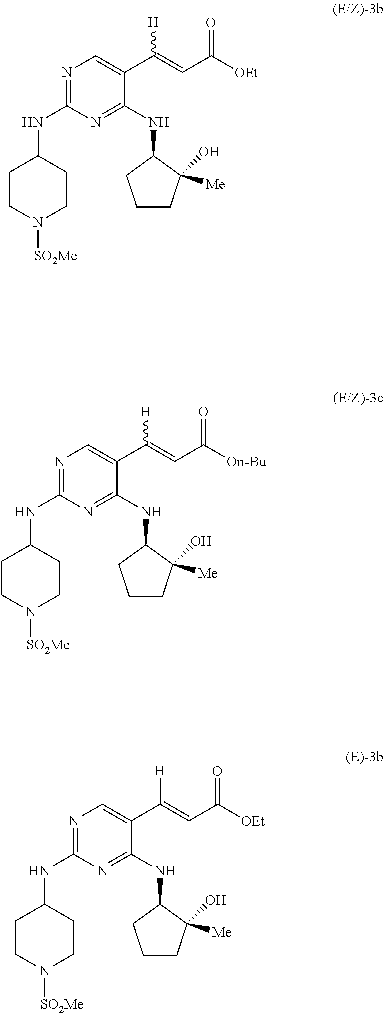

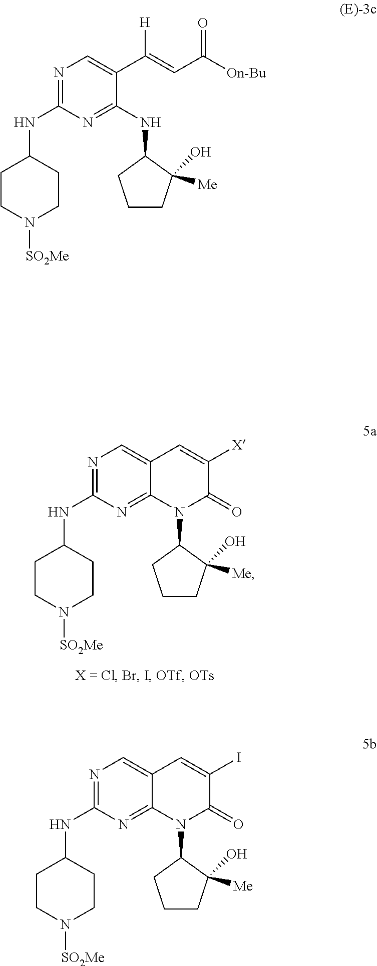

In one such embodiment, the invention provides a compound of Formula 3a:

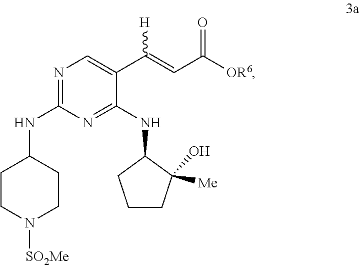

where R⁶ is C₁-C₄ alkyl or benzyl.

In some such embodiments, R⁶ is ethyl. In other such embodiments, R⁶ is n-butyl.

In another embodiment, the invention provides a compound of Formula 5a:

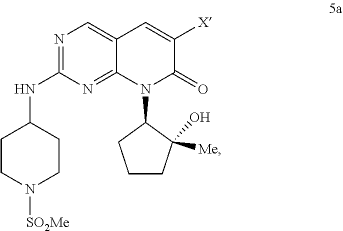

wherein X′ is Cl, Br, I, OTf or OTs.

In some such embodiments, X′ is I. In some such embodiments, X′ is Br. In some such embodiments, X′ is Cl. In some such embodiments, X′ is OTf or OTs.

In another aspect, the invention provides a method for preparing a compound of Formula 3a:

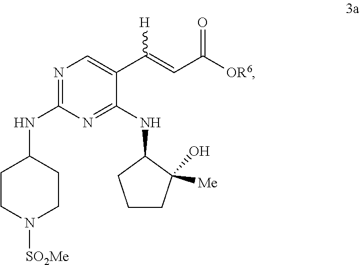

wherein R⁶ is C₁-C₄ alkyl or benzyl,

comprising treating the compound of Formula 2a:

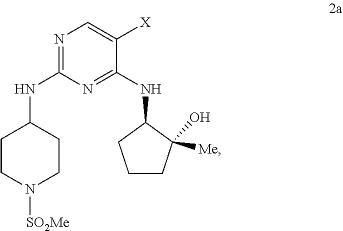

where X is Cl, Br, I, OTf or OTs,

with a C₁-C₄ alkyl acrylate or benzyl acrylate in the presence of a metal catalyst, such as a palladium catalyst, to provide the compound of Formula 3a.

In some embodiments, the reaction comprises reacting the compound of Formula 2a with ethyl acrylate to provide a compound of Formula 3a, wherein R⁶ is ethyl. In other embodiments, the reaction comprises reacting the compound of Formula 2a with n-butyl acrylate to provide a compound of Formula 3a, wherein R⁶ is n-butyl.

In particular embodiments, the metal catalyst is a palladium catalyst. In some such embodiments, the palladium catalyst is a palladium(II) catalyst. In specific embodiments, the palladium(II) catalyst is Pd(OAc)₂. In other such embodiments, the palladium catalyst is a palladium(0) catalyst. The palladium catalyst is typically present in an amount from about 0.01 to about 0.10 molar equivalents.

In some embodiments, the coupling reaction includes the presence of a ligand, such as a phosphine ligand. In some such embodiments, the phosphine ligand is selected from the group consisting of n-butyl-di-t-butylphosphonium tetraborofluorate, 1,4-bis(di-t-butylphosphonium)butane bis(tetrafluoroborate), triphenylphosphine, cyclohexyldiphenylphosphine, (oxydi-2,1-phenylene)-bis(diphenylphosphine) (DPEPhos), (oxydi-2,1-phenylene)bis(dicyclohexyl-phosphine) (DCyEPhos), 1,3-bis(diphenylphosphino)propane (dppp), 1,4-Bis(diphenyl-phosphino)-butane (dppb), di-(1-adamantyl)-n-butylphosphine (CataCXium® A), bis(di-tert-butyl(4-dimethylaminophenyl)phosphine (Amphos), 5-(di-tert-butylphosphino)-1′,3′,5′-triphenyl-1′H-[1,4′]bipyrazole (Bippyphos), 1,1′-bis(di-tert-butylphosphino)-ferrocene (DTBPF), 1,3-bis(2,6-diisopropylphenyl)imidazolinium chloride (SIPr—HCl) and 1,3-Bis(1-adamantyl)-4,5-dihydroimidazolium chloride (Sad-HCl). In specific embodiments, the phosphine ligand is n-butyl-di-t-butylphosphonium tetraborofluorate or (oxydi-2,1-phenylene)bis(diphenylphosphine) (DPEPhos). Where used, the phosphine ligand is typically present in an amount from about 0.01 to about 0.10 molar equivalents. In another aspect, the invention provides a method for preparing the compound of Formula 4:

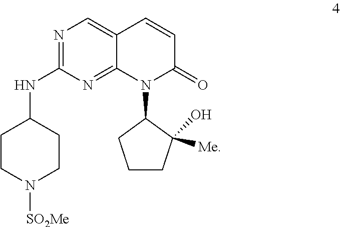 [SMILES: C[C@@]1(O)CCC[C@H]1n1c(=O)ccc2cnc(NC3CCN(S(C)(=O)=O)CC3)nc21]

comprising treating a compound of Formula 3a:

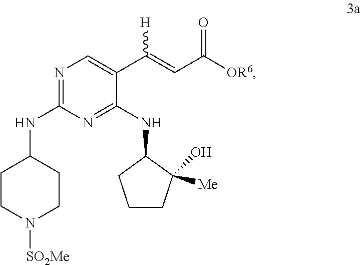

where R⁶ is C₁-C₄ alkyl or benzyl,

with a base to provide the compound of Formula 4.

In some embodiments, R⁶ is ethyl. In other embodiments, R⁶ is n-butyl.

In certain embodiments, the base is an alkoxide base, as further described herein. In some such embodiments, the alkoxide base is potassium tert-butoxide.

In a further aspect, the invention provides a method for preparing the compound of Formula 5b:

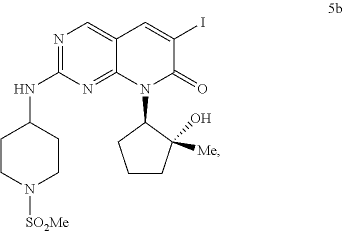 [SMILES: C[C@@]1(O)CCC[C@H]1n1c(=O)c(I)cc2cnc(NC3CCN(S(C)(=O)=O)CC3)nc21]

comprising treating the compound of Formula 4:

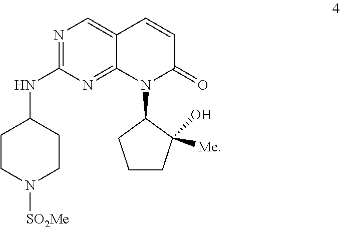 [SMILES: C[C@@]1(O)CCC[C@H]1n1c(=O)ccc2cnc(NC3CCN(S(C)(=O)=O)CC3)nc21]

with iodine or N-iodosuccinimide to provide the compound of Formula 5b.

In a further aspect, the invention provides a method for preparing the compound of Formula 5a:

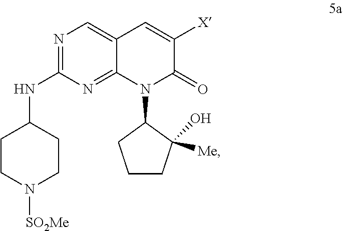

where X′ is Br, comprising treating the compound of Formula 4:

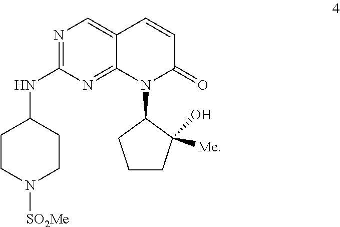 [SMILES: C[C@@]1(O)CCC[C@H]1n1c(=O)ccc2cnc(NC3CCN(S(C)(=O)=O)CC3)nc21]

with bromine or N-bromosuccinimide to provide the compound of Formula 5a where X′ is Br.

In some embodiments, the iodination or bromination reactions to provide 5b or 5a, wherein X′ is Br, are carried out in a polar aprotic solvent. In some such embodiments, the solvent is acetonitrile. In some embodiments, the iodination or bromination reaction is carried out in the presence of a protic source. In certain embodiments, the protic source is p-toluenesulfonic acid or oxalic acid. In some such embodiments, the protic source is present in catalytic or substoichiometric amounts.

Those skilled in the art will appreciate that modifications may be made to the synthetic routes described herein. Although specific starting materials and reagents are depicted in the schemes and Examples, other starting materials and reagents can be substituted to provide a variety of derivatives and/or reaction conditions. In addition, many of the compounds prepared by the methods described below can be further modified in light of this disclosure using conventional chemistry known to those of skill in the art.

### EXAMPLES

All reactions were performed under a nitrogen atmosphere. All reagents purchased from vendors were used as received unless specified otherwise. NMR data was collected using a Bruker AV III 400 MHz or a Bruker 600 MHz spectrometer with TCI cryoprobe. HRMS data was obtained using a Thermo Orbitrap XL using Electrospray Ionization in positive mode.

### Example 1

### Preparation of Ethyl (E)-3-(4-(((1R,2R)-2-hydroxy-2-methylcyclopentyl)amino)-2-((1-(methylsulfonyl)piperidin-4-yl)amino)pyrimidin-5-yl)acrylate (3b)

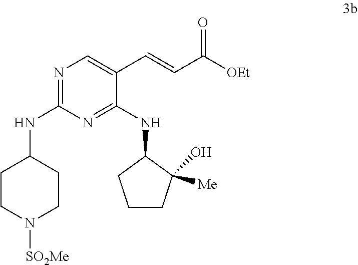 [SMILES: CCOC(=O)/C=C/c1cnc(NC2CCN(S(C)(=O)=O)CC2)nc1N[C@@H]1CCC[C@@]1(C)O]

(1R,2R)-2-((5-bromo-2-((1-(methylsulfonyl)piperidin-4-yl)amino)pyrimidin-4-yl)amino)-1-methylcyclopentan-1-ol (2b) (prepared as described in Example 7/Example 8 of U.S. Pat. No. 10,233,188) (5 g, 11.2 mmol) and n-butanol (75 mL) were combined. Ethyl acrylate (1.67 g, 16.7 mmol) was charged, followed by N, N-diisopropylethylamine (3.03 g, 23.4 mmol). The resulting mixture was degassed with vacuum and then purged with nitrogen (3 cycles). Palladium acetate (0.125 g, 0.558 mmol) and n-butyl-di(tert-butyl)phosphonium tetrafluoroborate (0.198 g, 0.669 mmol) were added. The reaction was heated to 95° C. and stirred at this temperature until the reaction was completed. After the reaction was cooled to ambient temperature, the reaction mixture was filtered through a CELITE® pad and the filter cake was washed with ethyl acetate (50 mL). The filtrate was washed with water and concentrated. The crude product was purified by flash chromatography to afford ethyl (E)-3-(4-(((1R,2R)-2-hydroxy-2-methylcyclopentyl)-amino)-2-((1-(methylsulfonyl)piperidin-4-yl)amino)pyrimidin-5-yl)acrylate (3b) (3.45 g, 7.82 mmol, 66% yield). Alternately, the crude, partially concentrated solution of 3b may be used directly without chromatography.

¹H NMR (400 MHz, DMSO-d₆) δ 8.28 (s, 1H), 7.80 (d, J=15.6 Hz, 1H), 7.15 (bd, J=27.3 Hz, 1H), 6.87 (d, J=7.6 Hz, 1H), 6.24 (d, J=15.6 Hz, 1H), 4.69 (bd, J=49.8 Hz, 1H), 4.36 (bs, 1H), 4.15 (q, J=7.1 Hz, 2H), 3.86 (bs, 1H), 3.58-3.47 (m, 2H), 2.90-2.78 (m, 1H), 2.87 (s, 3H), 2.05 (bs, 1H), 1.98-1.87 (m, 3H), 1.73-1.58 (m, 6H), 1.60-1.43 (m, 1H), 1.24 (t, J=7.1 Hz, 3H), 1.07 (s, 3H).

LRMS-ESI (m/z) [M+H]⁺ calcd for C₂₁H₃₃N₅O₅S, 468.22, found 468.49.

### Example 2

### Preparation of n-butyl (E)-3-(4-(((1R,2R)-2-hydroxy-2-methylcyclopentyl)amino)-2-((1-(methylsulfonyl)piperidin-4-yl)amino)pyrimidin-5-yl)acrylate (3c)

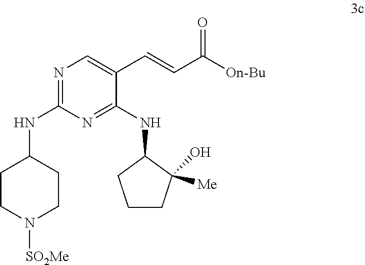

(1R,2R)-2-((5-bromo-2-((1-(methylsulfonyl)piperidin-4-yl)amino)pyrimidin-4-yl)amino)-1-methylcyclopentan-1-ol (2b) (prepared as described in Example 7/Example 8 of U.S. Pat. No. 10,233,188) (40.0 Kg, 89.2 mol), n-butanol (324 Kg) and water (1.60 L) were combined. Butyl acrylate (34.3 Kg, 268 mmol) was charged, followed by sodium bicarbonate (22.5 Kg, 268 mol). The resulting mixture was degassed with vacuum and then purged with nitrogen (2 cycles). Palladium acetate (401 g, 1.80 mol) and bis(2-diphenylphosphinophenyl)ether (1.20 Kg, 2.20 mol) were added. The vessel was subjected to pressure inertion to minimize oxygen content (4 cycles). The reaction was heated to 95° C. and stirred at this temperature until the reaction was complete. Upon completion, the mixture was cooled to 75° C., n-butanol (113 Kg) was added, the reaction mixture was filtered through a CELITE® pad and the filter cake was washed with n-butanol (2×65 Kg). The combined filtrates were concentrated to approximately 270 L and tert-butyl methyl ether (82.9 Kg) was added at 55° C. The resulting mixture was stirred at 55° for 2 hours, cooled to 10° C. over 4 hours and stirred for an additional 8 hours. The product was isolated by filtration, washed with tert-butyl methyl ether (59.2 Kg) and dried to afford butyl (E)-3-(4-(((1R,2R)-2-hydroxy-2-methylcyclopentyl)amino)-2-((1-(methylsulfonyl)-piperidin-4-yl)amino)pyrimidin-5-yl)acrylate (3c) (34.8 Kg, 77.1 mol, 86% yield).

(E/Z) mixture: ¹H NMR (600 MHz, DMSO-d₆, 338K): δ 8.23 (s, 0.8H), 8.19 (s, 0.2H), 7.75 (d, J=15.6 Hz, 0.8H), 6.95 (d, J=7.4 Hz, 0.8H), 6.90 (d, J=12.1 Hz, 0.2H), 6.78 (d, J=7.2 Hz, 0.2H), 6.61 (d, J=7.4 Hz, 0.8H), 6.20 (d, J=15.6 Hz, 0.8H), 6.16 (d, J=7.4 Hz, 0.2H), 5.71 (d, J=12.1 Hz, 0.2H), 4.61 (broad s, 0.2H), 4.56 (broad s, 0.8H), 4.35 (q, J=7.5 Hz, 0.8H), 4.32 (q, J=7.4 Hz, 0.2H), 4.11 (t, J=6.7 Hz, 1.6H), 4.05 (t, J=6.6 Hz, 0.4H), 3.92-3.84 (broad, 1H), 3.56 (m, 2H), 2.90-2.83 (m, 2H), 2.85 (s, 3H), 2.11-2.05 (broad, 1H), 1.99-1.92 (broad, 2H), 1.73-1.51 (m, 9H), 1.38 (m, 1.6H), 1.31 (m, 0.4H), 1.10 (s, 2.4H), 1.08 (s, 0.6H), 0.92 (t, J=7.5 Hz, 2.4H), 0.88 (t, J=7.4 Hz, 0.6H);

¹³C NMR (150 MHz, DMSO-d₆, 338K): δ 166.6, 165.9*, 161.0, 160.6*, 160.0, 159.9*, 157.6*, 156.1, 138.1, 136.8*, 114.9*, 110.9, 102.4*, 102.3, 79.1^{#}, 62.9*, 62.9, 60.7, 60.7*, 46.7, 46.6*, 44.2^{#}, 39.4^{#}, 34.5*, 30.8*, 30.7, 30.7*, 30.6, 30.2, 30.0*, 29.9, 29.8*, 23.4, 23.3*, 20.2, 20.1*, 18.4, 18.4*13.2, 13.2*;

*=Z-isomer; *=E and Z are overlapped
(E)-isomer (3c): ¹H NMR (600 MHz, DMSO-d₆) δ 0.90 (t, J=7.34 Hz, 3H), 1.06 (s, 3H), 1.32-1.40 (m, 2H), 1.44-1.56 (m, 2H), 1.56-1.72 (m, 7H), 1.84-2.14 (m, 3H), 2.79-2.85 (m, 2H), 2.86 (s, 3H), 3.45-3.60 (m, 2H), 3.75-3.96 (m, 1H), 4.10 (t, J=6.75 Hz, 2H), 4.25-4.46 (m, 1H), 4.55-4.82 (m, 1H), 6.24 (d, J=15.6 Hz, 1H), 6.86 (br d, J=7.34 Hz, 1H), 7.02-7.29 (m, 1H), 7.79 (d, J=15.6 Hz, 1H), 8.28 (s, 1H).

HRMS-HESI (m/z) [M+H]⁺ calcd for C₂₃H₃₈N₅O₅S⁺, 496.2588, found 496.2592.

### Example 3

### Preparation of 8-((1R,2R)-2-hydroxy-2-methylcyclopentyl)-2-((1-(methylsulfonyl)piperidin-4-yl)amino)pyrido[2,3-d]pyrimidin-7(8H)-one (4)

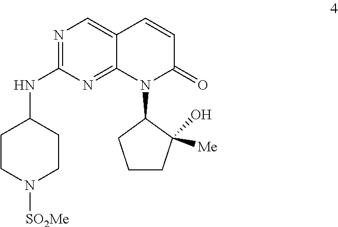 [SMILES: C[C@@]1(O)CCC[C@H]1n1c(=O)ccc2cnc(NC3CCN(S(C)(=O)=O)CC3)nc21]

### Method A: Preparation of Compound 4 Through 3b

Ethyl (E)-3-(4-(((1R,2R)-2-hydroxy-2-methylcyclopentyl)amino)-2-((1-(methyl-sulfonyl)piperidin-4-yl)amino)pyrimidin-5-yl)acrylate (3b) (4.5 g, 9.62 mmol) and tetrahydrofuran (27 mL) were combined. Potassium t-butoxide in tetrahydrofuran (1 mol/L, 38.5 mL, 38.5 mmol) was added at a temperature of 20° C. The reaction was heated at 45° C. until the reaction was complete. After cooling to ambient temperature, the reaction was quenched with water (50 mL) and diluted with ethyl acetate (200 mL). The layers were separated, and the organic phase was washed with brine. After concentration in vacuo, the crude solution was crystallized in a mixture of ethyl acetate/heptane to provide 8-((1R,2R)-2-hydroxy-2-methylcyclopentyl)-2-((1-(methylsulfonyl)piperidin-4-yl)amino)pyrido[2,3-d]pyrimidin-7(8H)-one (4) (2.1 g, 4.98 mmol, 52% yield).

### Method B: Preparation of Compound 4 Through 3c

n-Butyl-(E)-3-(4-(((1R,2R)-2-hydroxy-2-methylcyclopentyl)amino)-2-((1-(methylsulfonyl)piperidin-4-yl)amino)pyrimidin-5-yl)acrylate (3c) (32.67 Kg, 65.9 mol) and anhydrous tetrahydrofuran (281 Kg) were combined and heated to 50° C. Sodium sulfate (32.7 Kg, 230 mol) was added and the reaction mixture was heated to 60° C. A solution of 1M potassium tert-butoxide in tetrahydrofuran (88.8 kg, 98.9 mol) was added over two hours and then the mixture was stirred until the reaction was complete. The reaction mixture was cooled to 20° C., toluene (283 Kg) and water (327 Kg) were added and the mixture was stirred. The phases were separated, and the organic layer was concentrated until less than 5% THF remained in the toluene product mixture, replacing solvent with toluene as necessary. The resulting slurry was stirred at 10° C. The product was isolated by filtration, washed with toluene (2×56.6 Kg) and then dried to afford 8-((1R,2R)-2-hydroxy-2-methylcyclopentyl)-2-((1-(methylsulfonyl)piperidin-4-yl)amino)pyrido[2,3-d]pyrimidin-7(8H)-one (4) (26.54 Kg g, 62.9 mol, 95% yield).

The material was consistent with the compound prepared according to the procedure in Example 2 of U.S. Pat. No. 10,233,188.

### Example 4

### Preparation of 8-((1R,2R)-2-hydroxy-2-methylcyclopentyl)-6-iodo-2-((1-(methylsulfonyl)piperidin-4-yl)amino)pyrido[2,3-d]pyrimidin-7(8H)-one (5b)

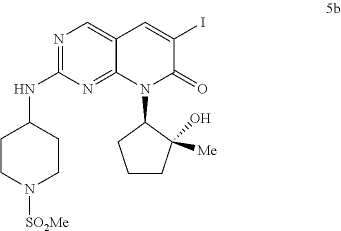 [SMILES: C[C@@]1(O)CCC[C@H]1n1c(=O)c(I)cc2cnc(NC3CCN(S(C)(=O)=O)CC3)nc21]

8-((1R,2R)-2-hydroxy-2-methylcyclopentyl)-2-((1-(methylsulfonyl)piperidin-4-yl)amino)pyrido[2,3-d]pyrimidin-7(8H)-one (4) (7.5 g, 17.79 mmol) and N-iodosuccinimide (6.0 g, 26.66 mmol) were charged to a 200 mL reactor. Acetonitrile (75 mL) was charged and the reactor was then sealed and purged with nitrogen. The mixture was agitated at 25-30° C. for about thirty minutes. After 30 minutes, the reactor was opened to air, and p-toluenesulfonic acid hydrate (0.35 g, 1.83 mmol) was added. The reactor was sealed, blanketed with nitrogen, and agitated for about two hours at 30° C. until the reaction reached about 95% conversion by UPLC. After two hours, the reaction was quenched with 5% sodium sulfite in water (5% w/w, 150 mL). Acetonitrile was distilled down to a final volume of 150 mL. The reaction was cooled to about 0° C. over 15 min and agitated for about one hour. The mixture was filtered over a Büchner funnel with filter paper under vacuum and washed twice with 5% acetonitrile in water (3 volumes each wash). The resulting wet cake was dried under vacuum oven at 50° C. to afford 7.4 g of 8-((1R,2R)-2-hydroxy-2-methylcyclopentyl)-6-iodo-2-((1-(methylsulfonyl)piperidin-4-yl)amino)pyrido[2,3-d]pyrimidin-7(8H)-one (5b) (76.7% yield, 99.9% purity).

¹H NMR (600 MHz, DMSO-d₆) δ: 8.63 (s, 0.35H), 8.59 (s, 0.65H), 8.45 (s, 1H), 8.05 (d, J=7.2 Hz, 0.65H), 7.82 (broad, 0.35H), 5.94-5.87 (m, 1H), 5.76 (s, 0.9H, CH₂Cl₂), 4.39 (broad, 0.65H), 4.35 (broad, 0.35H), 4.02 (broad, 0.35H), 3.89 (broad, 0.65H), 3.63-3.50 (m, 2H), 2.89-2.80 (m, 2H), 2.89 (s, 3H), 2.45-2.24 (broad, 1H), 2.24-2.13 (broad, 1.65H), 2.00-1.77 (m, 4.35H), 1.71-1.55 (m, 2.35H), 1.47 (m, 0.65H), 0.97 (s, 1.05H), 0.94 (s, 1.95H).

¹³C NMR (150 MHz, DMSO-d₆) δ: 160.2, 159.6, 158.6, (158.5), (156.4), 156.2, 145.2, (107.2), 106.5, (87.8), 87.5, (80.4), 80.3, 64.0, (63.5), 54.8 (CH₂Cl₂), 47.4, (47.2), (44.5), 44.3, 41.8, (41.7), 34.4, (34.1), (30.7), (30.6), 30.2, 30.1, (27.2), 26.7, 23.7, 23.3. The chemical shifts for a minor rotamer are listed in parenthesis.

### Example 5

### Preparation of 6-(difluoromethyl)-8-((1R,2R)-2-hydroxy-2-methylcyclopentyl)-2-((1-(methylsulfonyl)piperidin-4-yl)amino)pyrido[2,3-d]pyrimidin-7(8H)-one (1)

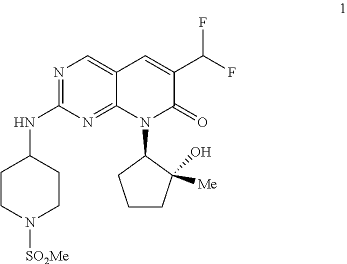 [SMILES: C[C@@]1(O)CCC[C@H]1n1c(=O)c(C(F)F)cc2cnc(NC3CCN(S(C)(=O)=O)CC3)nc21]

### Method A: Copper Difluoromethyl Complex

To an appropriate reaction vessel (A) was charged potassium tert-butoxide (2.26 g, 19.7 mmol) and copper(I) chloride (977 mg, 9.9 mmol). Dimethylformamide (14.4 mL) was added, and the mixture was stirred for 15 minutes at 20-30° C. Trimethylsilyl difluoromethane (2.74 mL, 20.1 mmol) was added, and the resulting mixture was stirred for 30 minutes at 20-30° C. A solution of 8-((1R,2R)-2-hydroxy-2-methylcyclopentyl)-6-iodo-2-((1-(methylsulfonyl)piperidin-4-yl)amino)pyrido[2,3-d]pyrimidin-7(8H)-one (5b) (4.0 g, 6.6 mmol) and propylene glycol (0.36 mL, 4.9 mmol) in dimethylformamide (11.2 mL) was charged to the mixture, which was stirred at 20-30° C. ° C. for 16 h.

Into a separate vessel (B) was charged potassium tert-butoxide (2.26 g, 19.7 mmol) and copper(I) chloride (977 mg, 9.9 mmol). Dimethylformamide (14.4 mL) was added, and the mixture was stirred for 15 minutes at 20-30° C. Trimethylsilyl difluoromethane (2.74 mL, 20.1 mmol) was added, and the resulting mixture was stirred for 30 minutes at 20-30° C. The mixture in vessel B was transferred to vessel A, and the resulting mixture was stirred for an additional 20-72 h.

The reaction mixture was transferred using 2-methyltetrahydrofuran (40 mL) to a reactor containing saturated aqueous ammonium chloride (20 mL) and aqueous magnesium chloride 35% w/w (20 mL). After stirring for 30 minutes, the layers were separated, and the aqueous phase was back extracted with 2-methyltetrahydrofuran (20 mL). Toluene (20 mL) was added to the combined organics and they were washed with saturated aqueous ammonium chloride (2×40 mL) and water (20 mL). The resulting organics were filtered through CELITE®, then the solvent was exchanged to toluene under vacuum and crystallization provided 6-(difluoromethyl)-8-((1R,2R)-2-hydroxy-2-methylcyclopentyl)-2-((1-(methylsulfonyl)piperidin-4-yl)amino)pyrido[2,3-d]pyrimidin-7(8H)-one (1) as an off-white solid (3.17 g, 89% yield).

### Example 6

### Preparation of 6-(difluoromethyl)-8-((1R,2R)-2-hydroxy-2-methylcyclopentyl)-2-((1-(methylsulfonyl)piperidin-4-yl)amino)pyrido[2,3-d]pyrimidin-7(8H)-one (1)

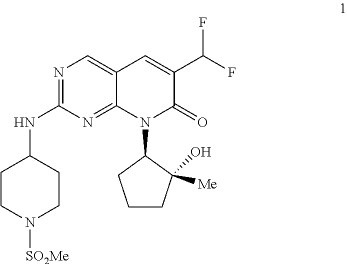 [SMILES: C[C@@]1(O)CCC[C@H]1n1c(=O)c(C(F)F)cc2cnc(NC3CCN(S(C)(=O)=O)CC3)nc21]

### Method B: Zinc Difluoromethyl Complex

An inerted clean reactor was charged with 8-((1R,2R)-2-hydroxy-2-methylcyclopentyl)-6-iodo-2-((1-(methylsulfonyl)piperidin-4-yl)amino)pyrido[2,3-d]pyrimidin-7(8H)-one (5b) (5.0 g, 8.312 mmol, 91 mass %). The reactor was evacuated and backfilled with nitrogen three times. Nitrogen sparged DMPU (40 mL) was charged followed by propylene glycol (0.25 equiv., 2.078 mmol, 100 mass %) or p-toluenesulfinic acid (0.25 equiv). The mixture was stirred until full dissolution was observed (15 min). A solution of copper(II) trifluoromethanesulfonate (0.9 equiv., 7.481 mmol, 98 mass %) in DMPU (40 mL, deep green) was charged to the reactor. The resulting green-yellow clear solution was stirred for 10-15 min at room temperature. A solution of Zn(CHF₂)₂(DMPU)₂ (3.0 equiv., 24.94 mmol, 78.76 mass %) in DMPU (20 mL, clear) was charged. The resulting reaction mixture was stirred over 24 h at room temperature and then sampled. Upon reaction completion, the in situ assay yield was determined to be 90-96%. Water was charged to quench excess zinc reagent and the mixture was diluted with Toluene/EtOAc (2:1). Aqueous NH₄OH was charged to make up a 10% aqueous solution. The layers were then separated. The organic layer was then washed with 10% NH₄Cl followed by water and 10% aq. NaCl. The solvent was distilled down to 10% at 50° C., and the desired product started to crystallize out. The mixture was allowed to cool down overnight and then filtered and dried to isolate 6-(difluoromethyl)-8-[(1R,2R)-2-hydroxy-2-methyl-cyclopentyl]-2-[(1-methylsulfonyl-4-piperidyl)amino]pyrido[2,3-d]pyrimidin-7-one (1) as an off-white solid (80-87% yield).

### Example 7

### Preparation of Zn(DMPU)₂(CHF₂)₂ by Continuous Process

The reactor system was first inerted with argon sweep. To the 300-mL jacketed reactor (as a continuous stirred-tank reactor, CSTR) under argon was added Zn(DMPU)₂(CHF₂)₂ (1.0 g, seed crystals prepared by the same process at small scale without seeding), followed by hexane (20 mL). With agitation on, CF₂HI stock solution (0.392 M in hexane), Et₂Zn in hexane solution (1.0 M) and neat DMPU were pumped into the CSTR concurrently, with a flow rate at 1.40 mmol/min, 0.70 mmol/min, and 1.45 mmol/min respectively. When the fill volume reached 200 mL, the slurry was transferred to a receiving reactor using a peristaltic pump adapted with PTFE tubing head at a 20 second on (at 600 rpm), 5 min off intermittent pumping cycles. The pumping was stopped after 442 min run time. The slurry in the receiver was filtered and the filter cake was washed with hexanes 3 times and dried under argon flow until a constant weight was obtained. In total, 121 g white powder was obtained (92% yield). The quantitative ¹⁹F NMR assay (in C₆D₆) was 91.0 wt %.

1. A method for preparing the compound of Formula 5a: 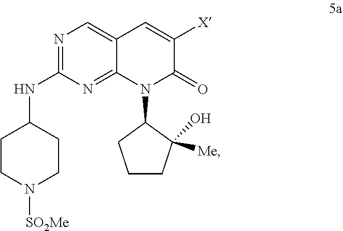
where X′ is Br or I,
comprising treating the compound of Formula 4: 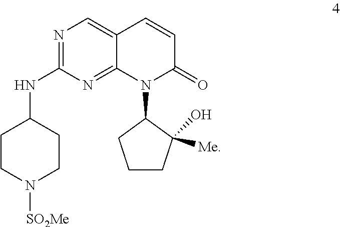 [SMILES: C[C@@]1(O)CCC[C@H]1n1c(=O)ccc2cnc(NC3CCN(S(C)(=O)=O)CC3)nc21]
(i) with bromine or N-bromosuccinimide to provide the compound of Formula 5a where X′ is Br; or (ii) with iodine or N-iodosuccinimide to provide the compound of Formula 5a where X′ is I.

2. The method of claim 1 for preparing the compound of Formula 5b: 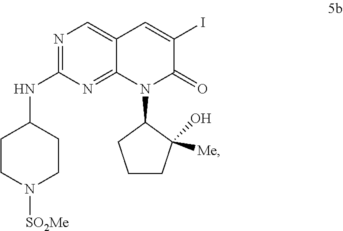 [SMILES: C[C@@]1(O)CCC[C@H]1n1c(=O)c(I)cc2cnc(NC3CCN(S(C)(=O)=O)CC3)nc21]
comprising treating the compound of Formula 4: 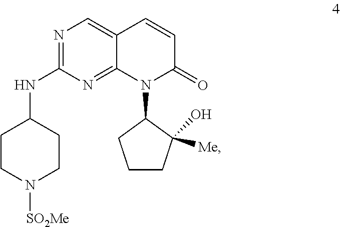 [SMILES: C[C@@]1(O)CCC[C@H]1n1c(=O)ccc2cnc(NC3CCN(S(C)(=O)=O)CC3)nc21]
with iodine or N-iodosuccinimide to provide the compound of Formula 5b.

3. The method of claim 1, further comprising a protic source.

4. The method of claim 3, wherein the protic source is p-toluenesulfonic acid.

5. A method for preparing the compound of Formula 4: 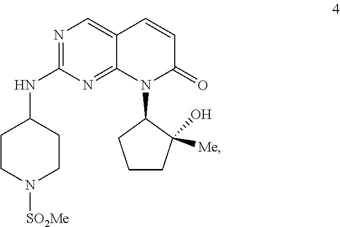 [SMILES: C[C@@]1(O)CCC[C@H]1n1c(=O)ccc2cnc(NC3CCN(S(C)(=O)=O)CC3)nc21]
comprising treating a compound of Formula 3a: 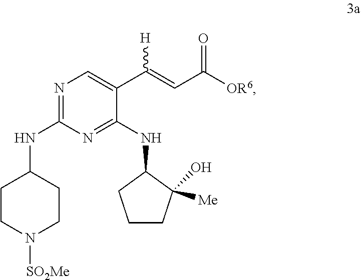
where R⁶ is C₁-C₄ alkyl or benzyl,
with a base to provide the compound of Formula 4.

6. The method of claim 5, wherein R⁶ is ethyl or n-butyl.

7. The method of claim 5, wherein the base is an alkoxide base.

8. A method for preparing a compound of Formula 3a: 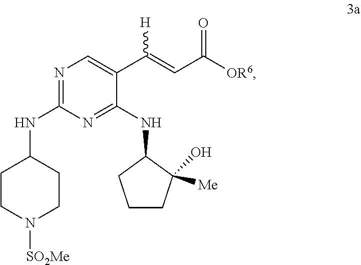
wherein R⁶ is C₁-C₄ alkyl or benzyl,
comprising treating the compound of Formula 2a: 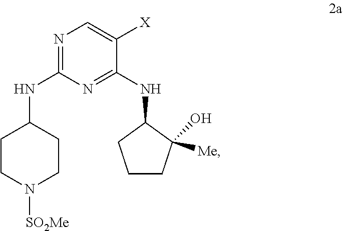
where X is Cl, Br, I, OTf or OTs,
with a C₁-C₄ alkyl acrylate or benzyl acrylate in the presence of a palladium catalyst to provide the compound of Formula 3a.

9. The method of claim 8, wherein R⁶ is ethyl or n-butyl.

10. The method of claim 8, wherein X is Br.

11. The method of claim 8, wherein the palladium catalyst is Pd(OAc)₂.

12. The method of claim 8, further comprising the presence of a phosphine ligand.

13. The method of claim 12, wherein the phosphine ligand is n-butyl-di-t-butylphosphonium tetraborofluorate or (oxydi-2,1-phenylene)bis(diphenylphosphine) (DPEPhos).

14. A method for preparing the compound of Formula 1: 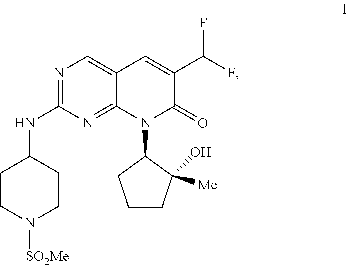 [SMILES: C[C@@]1(O)CCC[C@H]1n1c(=O)c(C(F)F)cc2cnc(NC3CCN(S(C)(=O)=O)CC3)nc21]
comprising reacting a compound of Formula 5a: 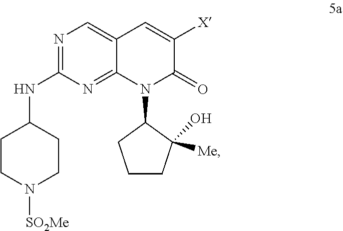
where X′ is Cl, Br, I, OTf or OTs,
with a difluoromethylation agent and a copper reagent to provide the compound of Formula 1.

15. The method of claim 14, wherein the difluoromethylation agent is a difluoromethyltrialkylsilane.

16. The method of claim 15, wherein the difluoromethyltrialkylsilane is difluoromethyltrimethylsilane (TMSCHF₂).

17. The method of claim 14, wherein the difluoromethylation agent is a zinc difluoromethyl complex.

18. The method of claim 17, wherein the zinc difluoromethyl complex is Zn(DMPU)₂(CHF₂)₂.

19. The method of claim 14 for preparing the compound of Formula 1, 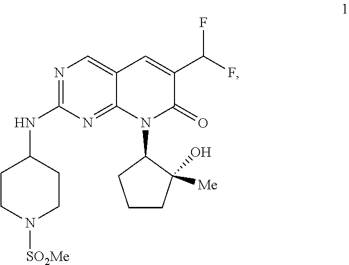 [SMILES: C[C@@]1(O)CCC[C@H]1n1c(=O)c(C(F)F)cc2cnc(NC3CCN(S(C)(=O)=O)CC3)nc21]
comprising reacting the compound of Formula 5b: 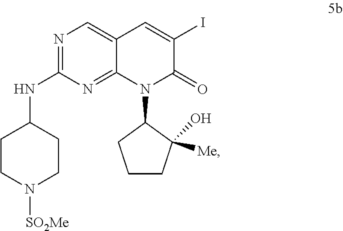 [SMILES: C[C@@]1(O)CCC[C@H]1n1c(=O)c(I)cc2cnc(NC3CCN(S(C)(=O)=O)CC3)nc21]
with a difluoromethyltrialkylsilane, a copper reagent and a base, to provide the compound of Formula 1.

20. The method of claim 19, wherein the difluoromethyltrialkylsilane is TMSCHF₂.

21. The method of claim 19, wherein the base is an alkoxide base.

22. The method of claim 19, further comprising a protic source.

23. The method of claim 22, wherein the protic source is p-toluenesulfinic acid, water, propylene glycol or pinacol.

24. The method of claim 14 for preparing the compound of Formula 1, 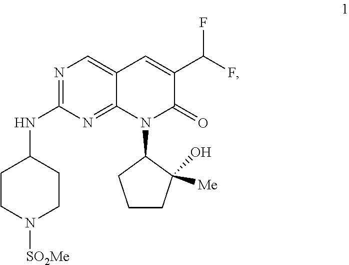 [SMILES: C[C@@]1(O)CCC[C@H]1n1c(=O)c(C(F)F)cc2cnc(NC3CCN(S(C)(=O)=O)CC3)nc21]
comprising reacting a compound of Formula 5b: 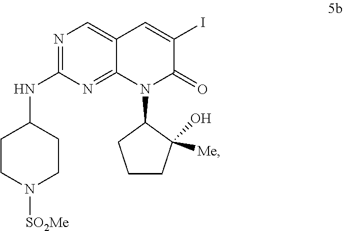 [SMILES: C[C@@]1(O)CCC[C@H]1n1c(=O)c(I)cc2cnc(NC3CCN(S(C)(=O)=O)CC3)nc21]
with a zinc difluoromethyl complex and a copper reagent to provide the compound of Formula 1.

25. The method of claim 24, wherein the zinc difluoromethyl complex is Zn(DMPU)₂(CHF₂)₂.

26. The method of claim 24 or 25, further comprising a protic source.

27. The method of claim 26, wherein the protic source is p-toluenesulfinic acid, water, propylene glycol or pinacol.

28. The method of claim 1 for preparing the compound of Formula 1, 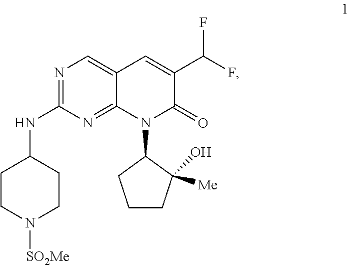 [SMILES: C[C@@]1(O)CCC[C@H]1n1c(=O)c(C(F)F)cc2cnc(NC3CCN(S(C)(=O)=O)CC3)nc21]
comprising reacting the compound of Formula 5b: 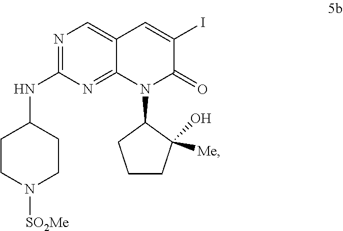 [SMILES: C[C@@]1(O)CCC[C@H]1n1c(=O)c(I)cc2cnc(NC3CCN(S(C)(=O)=O)CC3)nc21]
with continuously or semi-continuously prepared Zn(DMPU)₂(CHF₂)₂ and a copper reagent in a contiguous continuous or semi-continuous process.

29. The method of claim 14, wherein the copper reagent is CuCl, CuI, Cu(OTf), Cu(OTf)₂, Cu(BF₄)(MeCN)₄ or Cu(PF₆)(MeCN)₄.

30. (canceled)

31. A compound of Formula 5a: 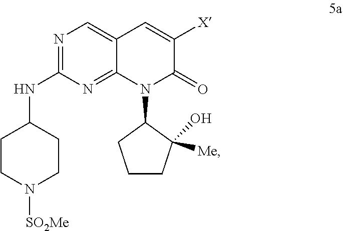
wherein X′ is Br, I, OTf or OTs.

32. The compound of claim 31, wherein X′ is I.

33. (canceled)

34. A compound of Formula 3a: 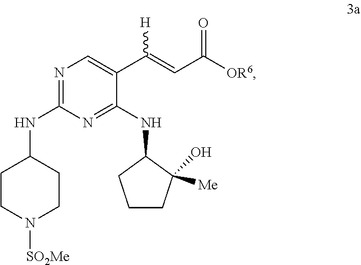
where R⁶ is C₁-C₄ alkyl or benzyl.

35. The compound of claim 34, wherein R⁶ is ethyl or n-butyl.

36. (canceled)

37. (canceled)
